(19) United States
(12) Patent Application Publication
(10) Pub. No.: US 2026/0053998 A1
(43) Pub. Date: Feb. 26, 2026

(54) CANNULA SYSTEM INCLUDING VARIABLE SHAPE TIP

(71) Applicant: Vitara Biomedical, Inc., Philadelphia, PA (US)

(72) Inventors: Christopher C. GREGORY, Newtown, PA (US); Jeff KIERSH, Exton, PA (US)

(73) Assignee: Vitara Biomedical, Inc., Philadelphia, PA (US)

(21) Appl. No.: 19/313,337

(22) Filed: Aug. 28, 2025

Related U.S. Application Data
(63) Continuation-in-part of application No. 19/303,890, filed on Aug. 19, 2025.
Application No. 19/303,890 is a continuation of application No. PCT/US2024/016401, filed on Feb. 19, 2024.
(60) Provisional application No. 63/447,017, filed on Feb. 20, 2023.

(51) Int. Cl. A61M 1/36; A61M 1/16

(52) U.S. Cl. CPC A61M 1/3659; A61M 1/1698; A61M 2205/0266; A61M 2210/12; A61M 2240/00

(57) ABSTRACT

A cannula system for cannulating a blood vessel includes a cannula having a distal end and a proximal end. The cannula defines a cannula lumen. The cannula system also includes a tip on the distal end of the cannula. The tip defines a tip lumen connected to the cannula lumen. The tip is variable between a first shape and a second shape. The first shape has a first width sized to fit in a lumen of the blood vessel. The second shape has a second width larger than the first width and sized to engage an inner surface of the blood vessel defining the lumen and secure the tip in the lumen of the blood vessel.

### CROSS-REFERENCE TO RELATED APPLICATIONS

This application is a continuation-in-part of U.S. Nonprovisional patent application Ser. No. 19/303,890, entitled “Cannula System Including Variable Shape Tip,” filed Aug. 19, 2025, which is a continuation of PCT/US2024/016401, filed Feb. 19, 2024, entitled “Cannula System Including Variable Shape Tip,” which claims priority to and the benefit of U.S. Provisional Application No. 63/447,017, filed Feb. 20, 2023, entitled “Cannula System Including Variable Shape Tip,” the disclosure of each of which is incorporated herein by reference in its entirety.

### BACKGROUND OF THE INVENTION

Extreme prematurity is the leading cause of infant morbidity and mortality in the United States for children under the age of five, with over one third of all infant deaths and one-half of cerebral palsy diagnoses attributed to prematurity. Respiratory failure represents the most common and challenging problem associated with extreme prematurity, as gas exchange in critically preterm neonates is impaired by structural and functional immaturity of the lungs. Advances in neonatal intensive care have achieved improved survival and pushed the limits of viability of preterm neonates from about 23 to about 24 weeks gestation, which marks the transition from the canalicular to the saccular phase of lung development. Although survival has become possible, there is still a high rate of chronic lung disease and other complications of organ immaturity, particularly in neonates born prior to 28 weeks gestation. The development of a system that could support normal neonatal growth and organ maturation for even a few weeks could significantly reduce the morbidity and mortality of extreme prematurity and improve quality of life in survivors.

Cannula systems can facilitate cannulation of veins, arteries, and the like, of a patient such as a neonate. Cannula systems typically include a trocar or needle for puncturing a patient body and a cannula for insertion into the patient via the puncturing. In addition, some cannula systems may include a dilator that is used to position the cannula. The needle and dilator must be inserted into the cannula from a proximal position to extend out through the cannula tip a precise amount. During use, the needle is used to penetrate the vessel wall and advanced until the tip of the dilator passes through the opening. The needle is then retracted, and the dilator is further advanced until the cannula is within the vessel the proper depth and then the dilator is retracted. Simultaneously to the dilator retraction, a device must be engaged with the vessel to secure the cannula. The dilator must then be removed from the cannula to open the passage for blood. The relatively large outside diameter of the dilator requires a large opening in the side of the cannula to allow the dilator to pass into and out of the cannula lumen. In addition, the securement device is complex and results in a large amount of space around or within the cannula being occupied. As a result, the blood vessels may be unduly occupied and stretched. Also, there is a need to make the attachment between a cannula and anatomical vessel more secure.

### SUMMARY

Improved cannula systems are described herein. In one aspect, provided herein is a cannula system for cannulating a blood vessel. The cannula system includes a cannula having a distal end and a proximal end. The cannula defines a cannula lumen. The cannular system also includes a tip on the distal end of the cannula. The tip defines a tip lumen connected to the cannula lumen. The tip is variable between a first shape and a second shape. The first shape has a first width sized to fit in a lumen of the blood vessel. The second shape has a second width larger than the first width and sized to engage an inner surface of the blood vessel defining the lumen and secure the tip in the lumen of the blood vessel.

In another aspect, a method of cannulating a blood vessel in a tissue includes inserting a needle into the blood vessel to form a passage in the blood vessel. The needle extends through a tip on a distal end of a cannula, and the method further includes inserting the tip and the cannula through the passage and into a lumen defined by the blood vessel. The tip is switched from a first shape to a second shape. The first shape has a first width sized to fit in the lumen of the blood vessel. The second shape has a second width larger than the first width and sized to engage an inner surface of the blood vessel defining the lumen and secure the tip in the lumen of the blood vessel.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts a schematic of an extracorporeal support system.

FIG. 2 depicts a schematic of a portion of an extracorporeal support system.

FIG. 3 depicts an isometric view of a portion of an extracorporeal support system.

FIG. 4 is a schematic illustration of a cannula system, according to an embodiment of the present disclosure.

FIGS. 5-7 are schematic illustrations of a cannula included in the cannula system of FIG. 4, and showing a variable tip of the cannula in a contracted configuration, a first expanded configuration, and a second expanded configuration, respectively.

FIG. 8 depicts a cannula system implementing a variable tip according to an embodiment of the present disclosure, with the variable tip in a first shape.

FIG. 9 depicts an isometric view of a portion of the cannula system of FIG. 4, with the variable tip in the second shape.

FIG. 10 depicts an enlarged isometric view of the variable tip of the cannula system of FIG. 4, and illustrating surface features on the variable tip.

### DETAILED DESCRIPTION OF THE INVENTION

Improved cannula systems are described herein. The cannula systems can be included in and/or used with extracorporeal support systems such as an extracorporeal support system configured to provide support for neonates. The cannula systems described herein include a tip having a variable shape. In some embodiments, the cannula systems can be implemented without the use of a dilator and can be simpler than prior systems. In addition, cannulas described herein may not use or require a slit port on a side of the cannula or a collet clamp. Also, the cannula systems simplify or eliminate a need for specialized tools and methods used to coordinate and position the cannula. Moreover, the cannula may have a reduced diameter and, therefore, be easier to position in smaller vessels than prior cannulas. In addition, described embodiments reduce the number of steps in methods of using a cannula and simplify the cannulation process.

In some embodiments, a cannula system for cannulating a blood vessel includes a cannula having a distal end and a proximal end, and defining a cannula lumen. The cannular system also includes a tip on the distal end of the cannula. The tip defines a tip lumen connected to the cannula lumen. The tip is variable between a first shape and a second shape. The first shape has a first width sized to fit in a lumen of the blood vessel. The second shape has a second width larger than the first width and sized to engage an inner surface of the blood vessel defining the lumen and secure the tip in the lumen of the blood vessel.

In some embodiments, a method of cannulating a blood vessel in a tissue includes inserting a needle into the blood vessel to form a passage in the blood vessel. The needle extends through a tip on a distal end of a cannula, and the method further includes inserting the tip and the cannula through the passage and into a lumen defined by the blood vessel. The tip is switched from a first shape to a second shape. The first shape has a first width sized to fit in the lumen of the blood vessel. The second shape has a second width larger than the first width and sized to engage an inner surface of the blood vessel defining the lumen and secure the tip in the lumen of the blood vessel.

In some embodiments, an extracorporeal system for supporting a neonate includes a chamber configured to receive a neonate, an oxygenation circuit having an oxygenator, and a cannula system for cannulating a blood vessel of the neonate. The cannula system includes a cannula and a tip coupled to a distal end of the cannula. The cannula defines a cannula lumen and the tip defines a tip lumen connected to the cannula lumen. The tip is variable between a first shape and a second shape. The first shape has a first width sized to fit in a lumen of the blood vessel. The second shape has a second width larger than the first width and sized to engage an inner surface of the blood vessel defining the lumen and secure the tip in the lumen of the blood vessel.

The terminology used herein is for the purpose of describing particular embodiments, implementations, and/or concepts (including any feature(s) or aspect(s) thereof) and is not intended to be limiting. Unless defined otherwise, all technical and/or scientific terms used herein have the same meanings as commonly understood by one of ordinary skill in the art. Any explanation or discussion of or using particular terms is intended to provide context and to facilitate understanding and is not necessarily intended to replace or supersede commonly used or known definitions understood by one skilled in the art unless explicitly stated otherwise. Moreover, various terms may be used to describe similar or substantially the same embodiments, implementations, and/or concepts (including any feature(s) or aspect(s) thereof) and thus, the use of particular terms is not intended to be limiting and/or to the exclusion of other terms unless the terms are mutually exclusive, or the context clearly states otherwise.

As used herein, the singular forms “a,” “an,” and “the” are intended to include the plural forms as well, unless the context clearly indicates otherwise. With respect to the use of singular and/or plural terms herein, those having skill in the art can translate from the singular to the plurality and/or vice versa as is appropriate for the context and/or application. Furthermore, any reference herein to a singular component, feature, aspect, etc. is not intended to imply the exclusion of more than one such component, feature, aspect, etc. (and/or vice versa) unless expressly stated otherwise.

As used herein, the terms “about,” “approximately,” and/or “substantially” when used in connection with stated value(s) and/or geometric structure(s) or relationship(s) is intended to convey that the value or characteristic so defined is nominally the value stated or characteristic described. For example, a first structure or feature may be described as being substantially parallel to a second structure or feature when the structures are nominally parallel. In some instances, the terms “about,” “approximately,” and/or “substantially” can generally mean and/or can generally contemplate a value or characteristic stated within a desirable or normal tolerance in the art (e.g., within two standard deviations, plus/minus 10% or less of the value or characteristic stated). While a value, structure, and/or relationship stated may be desirable, it should be understood that some variance may occur as a result of, for example, manufacturing tolerances or other practical considerations (such as, for example, applied pressures or forces, temperature variances, and/or the like). Accordingly, the terms “about,” “approximately,” and/or “substantially” can be used herein to account for such tolerances and/or considerations. Unless otherwise clear from context, all numerical values provided herein are modified by the term “approximately” or “about,”

In general, terms used herein and in the appended claims are intended as “open” terms unless expressly stated otherwise. For example, the term “including” should be interpreted as “including but not limited to,” the term “having” should be interpreted as “having at least,” etc. Similarly, the term “comprising” may specify the presence of stated features, elements, components, integers (or fractions thereof), steps, operations, and/or the like but does not preclude the presence or addition of one or more other features, elements, components, integers (or fractions thereof), steps, operations, elements, components, and/or groups thereof, and/or the like unless such combinations are otherwise mutually exclusive.

As used herein the term “and/or” includes any and all combinations of one or more of the associated listed items. It should be understood that any suitable disjunctive word and/or phrase presenting two or more alternative terms, whether in the written description or claims, contemplate the possibilities of including one of the terms, either of the terms, or both/all of the terms. For example, the phrase “A and/or B” will be understood to include the possibilities of “A” alone. “B” alone, or a combination of “A and B,” Unless specifically stated or otherwise clear from context, the term “or” as used herein, will be understood to be inclusive.

All ranges described herein include each individual member or value of the listed range, including the end members or values. Any listed ranges are intended to encompass any and all possible subranges and/or combinations of subranges thereof unless expressly stated otherwise. Any listed range should be recognized as sufficiently describing and enabling the same range being broken down into at least equal subparts unless expressly stated otherwise. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

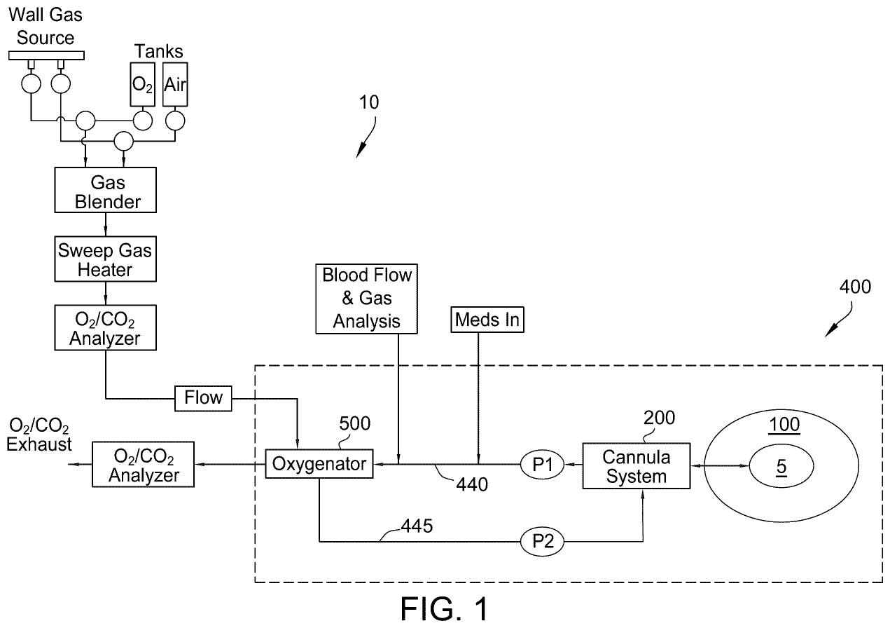

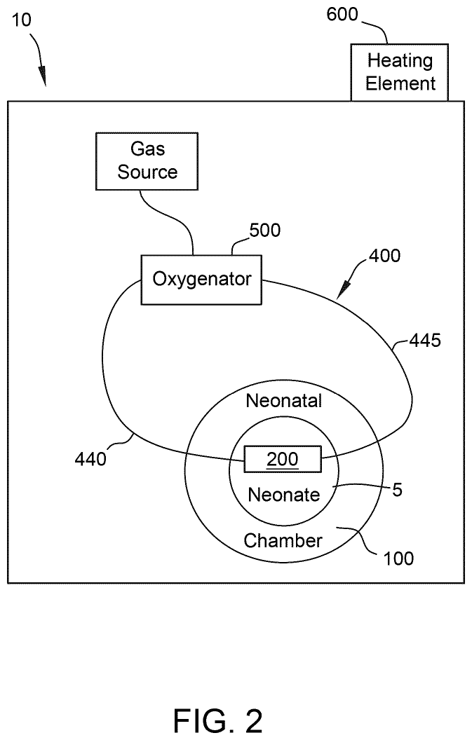

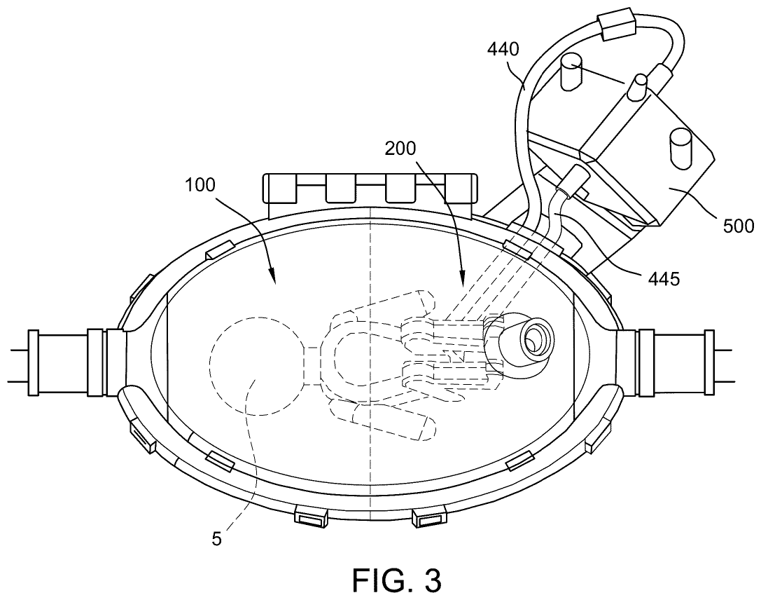

Referring to FIGS. 1-3, a system 10 is configured to provide extracorporeal support to a neonate 5. According to one aspect of the disclosure, the system 10 may be configured to provide an extracorporeal environment that is similar to an environment the neonate would experience in utero. Viability of a neonate that is removed from the uterine environment (e.g., due to preterm birth) and that is, for example, between about 23 weeks to about 24 weeks gestation may be increased by placing the neonate in the extracorporeal environment provided by the system 10.

According to an aspect of the disclosure, the system 10 may be configured to accomplish any one of the following: (1) limit exposure of the neonate 5 to light: (2) limit exposure of the neonate 5 to sound: (3) maintain the neonate 5 submerged within a liquid environment: (4) maintain the neonate 5 within a desired temperature range: (5) minimize exposure to environmental contaminants: or (5) any combination thereof. The system 10 also permits neonatal activities (e.g., neonatal breathing movements, neonatal swallowing of fluid) necessary for organ growth and development.

The system 10 may be configured to treat neonates (e.g., less than 37 weeks estimated gestational age, particularly 28 to 32 weeks estimated gestational age), or extreme premature neonates (23 to 28 weeks estimated gestational age). The gestation periods are provided for humans, though corresponding preterm neonates of other animals may be used. In a particular embodiment, the neonate has no underlying congenital disease. The term or preterm neonate may have limited capacity for pulmonary gas exchange, for example, due to pulmonary hypoplasia or a congenital anomaly affecting lung development, such as congenital diaphragmatic hernia. In a particular aspect, the subject may be a preterm or term neonate awaiting lung transplantation, for example, due to congenital pulmonary disease (e.g., bronchoalveolar dysplasia, surfactant protein B deficiency, and the like). Such transplantation surgeries are currently rarely performed in the United States. However, the number of transplantation surgeries may be increased with the more stable method for pulmonary support provided by embodiments of the disclosure. The neonate 5 may also be a candidate for ex utero intrapartum treatment (EXIT) delivery, including patients with severe airway lesions and a long-expected course before definitive resection. The neonate 5 may also be a neonatal surgical or fetoscopic procedure patient, particularly with preterm labor precipitating early delivery. According to one aspect of the disclosure, the system 10 may be configured such that the neonate 5 is maintained in the system 10 for as long as needed (for example, for days, weeks or months, until the neonate 5 is capable of life without the system 10). The system 10 should be operable to maintain the neonate 5 for at least 7 days, at least 14 days, at least 21 days, at least 28 days, at least 35 days, at least 42 days, at least 49 days, or at least 56 days.

The system 10 includes a neonatal chamber 100 configured to house a neonate 5, a physiologic saline solution (PSS) circuit configured to provide a flow (e.g., a constant flow) of PSS through the neonatal chamber 100, an oxygenation circuit 400 (e.g., extracorporeal membrane oxygenation system) configured to remove carbon dioxide from the neonate's blood and supply oxygen to the neonate's blood, and a cannula system 200 configured to establish communication between the vasculature of the neonate 5 and the oxygenation circuit 400.

The system 10 is configured to maintain the neonate 5 in the neonatal chamber 100 immersed in PSS. The system 10 is further configured such that the oxygenation circuit 400 provides adequate gas exchange for the neonate 5 to sustain life. In this way, the system 10 provides an environment similar to an intrauterine environment to facilitate continued growth and development of the neonate 5. The system 10 may include a cart or similar device that facilitates monitoring, caring for, and transporting the neonate 5 within a medical facility.

According to an aspect of this disclosure, the system 10 may be similar to or substantially the same as the systems described in U.S. Pat. No. 11,471,351, filed Jun. 13, 2019, entitled “System and Method Configured to Provide Extracorporeal Support for Premature Fetus,” the disclosure of which is incorporated herein by reference in its entirety.

The oxygenation circuit 400 can be connected with the neonate 5 via the cannula system 200 in a venous/venous arrangement (e.g., with a mechanical pump included in the system 10). Alternatively, the oxygenation circuit 400 may be connected with the neonate 5 via the cannula system 200 in an arterial/venous arrangement. The cannula system 200 can include one or more cannula(s) that may be placed in the great neck vessels (e.g., carotid, jugular) of the neonate 5 to connect the circulatory system of the neonate 5 to the oxygenation circuit 400. The placement of the one or more cannula(s) in the great neck vessels may avoid issues of vasospasm and/or cannula instability that may be encountered in umbilical vessels. In other implementations, however, the cannula system 200 can allow for cannulation of at least a vein or an artery of the umbilical cord of the neonate 5.

An external portion of the cannulas may be fitted with a sleeve (e.g., to permit increased tension of stabilizing sutures). The sleeve may be made of silicone and may be, for example, about 1-10 centimeters (cm) in length, and more particularly about 3-5 cm in length. The cannula(s) of the cannula system 200 may be sutured to the neonate 5 (for example via the fitted sleeve) to secure the cannulas to the neck of the neonate 5. Alternatively, in implementations in which the cannula system 200 connects the oxygenation circuit 400 to the neonate 5 via the neonate's umbilical cord, cannulas may be sutured into the vein(s) and/or artery (ies) of the umbilical cord. It will be appreciated that other connection arrangements may be utilized. For example, a non-suturing device is described in U.S. Patent Publication No. 2021/0338270 (“the '270 publication”), filed Apr. 28, 2021, entitled “Cannula Insertion System And Methods Of Using The Same” the disclosure of which is incorporated herein by reference in its entirety.

The oxygenation circuit 400 may include an oxygenator 500 for providing gas exchange functionality, particularly of oxygen (to) and carbon dioxide (from), to the neonate 5. The oxygenator 500 can be removably connected to the neonate 5 and, optionally, to other components of the oxygenation circuit 400 and the system 10. The oxygenator 500 includes two or more fluid lines that are connected to the neonate 5 via the cannula system 200. For example, the oxygenator 500 can include at least a drain line 440 and an inlet line 445 that are connected to the neonate 5 via the cannula system 200. In such implementations, blood flows from the neonate 5 though the drain line 440 to the oxygenator 500. The blood then flows through the oxygenator 500 and returns to the neonate 5 via the inlet line 445 (and the cannula system 200).

In some embodiments, the oxygenator 500 may be configured to be disconnected and replaced while the oxygenation circuit 400 is operational. If the oxygenator 500 is damaged or has surpassed its expected life cycle (typically 8 hours based on regulatory approvals), the oxygenation circuit 400 may be temporarily configurable to bypass the oxygenator 500 so that the oxygenator 500 may be disconnected from the oxygenation circuit 400 and a new, primed, oxygenator 500 connected in its place without interruption of blood flow.

A heating and/or cooling element 600 may be connected to the system 10 and arranged to regulate a temperature of one or more components of the system 10.

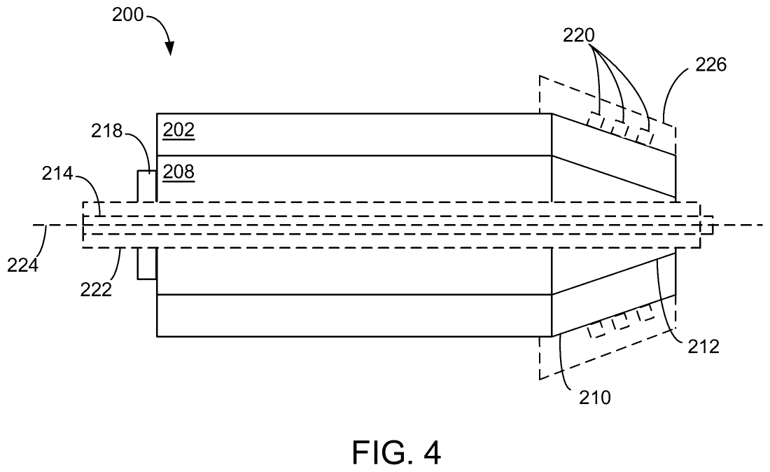

FIG. 4 is a schematic illustration of a cannula system 200 suitable for use in the extracorporeal support system 10 (and/or the oxygenation circuit 400 thereof) described above with reference to FIGS. 1-3, according to an embodiment. In some implementations, the cannula system 200 may be suitable for use with and/or may be integrated into a cannula insertion system described in detail in the '270 publication incorporated by reference herein. As described herein, the cannula system 200 can be arranged for cannulating blood vessels such as, for example, the blood vessels of an umbilical cord of a neonate patient. The cannula system 200 includes a cannula 202 having a cannula lumen 208 and a tip 210 having a tip lumen 212. Optionally, the cannula system 200 can further include features 220, a collet 226, a needle sheath 222, a needle 214, and a guidewire 224, as described in further detail herein.

The cannula 202 can be any suitable cannula that is shaped, sized, and/or configured for use in an extracorporeal support system such as the system 10 described above with reference to FIGS. 1-3. As such, the cannula 202 or at least a portion thereof can be sized to fit in the blood vessels of a pediatric (e.g., neonate) patient. For example, in some embodiments, the cannula 202 can be in a range between 24 gauge to 32 gauge. The cannula 202 defines a cannula lumen 208 that extends through the cannula 202 from a proximal end of the cannula 202 to a distal end of the cannula 202. The cannula 202 can include any number of sections, portions, regions, etc. having one or more set of desired characteristics. For example, the cannula 202 can include a rigid section extending proximally from the distal end of the cannula 202, and a flexible section extending from the proximal end of the cannula 202 (e.g., toward the rigid section). The cannula 202 can be composed of biocompatible materials such as stainless steel, cobalt chrome alloy, titanium, nickel-titanium alloy, tantalum, tungsten plated nickel, tungsten plated brass, polycarbonate, polyetheretherketone (PEEK), polyethersulfone (PES), thermoplastic elastomers (e.g., urethane), fiber-reinforced plastic, or any other suitable biocompatible material, and/or combination(s) thereof.

As shown in FIG. 4, the cannula 202 can include an access port 218 or the like that is configured to receive the needle 214. The access port 218 can be located along a portion of the cannula 202 such that the access port 218 is substantially aligned with at least a portion of the cannula lumen 208 and the tip lumen 212. For example, the access port 218, at least the portion of the cannula lumen 208, and the tip lumen 212 can be aligned in a manner that allows a relative rigid, straight needle 214 to be advanced through the cannula system 200. In some embodiments, the access port 218 can be a septum seal and/or any suitable port (e.g., a self-healing port) that can allow insertion of the needle 214 through the cannula system 200 and automatic or self-sealing of the access port 218 upon removal of the needle 214.

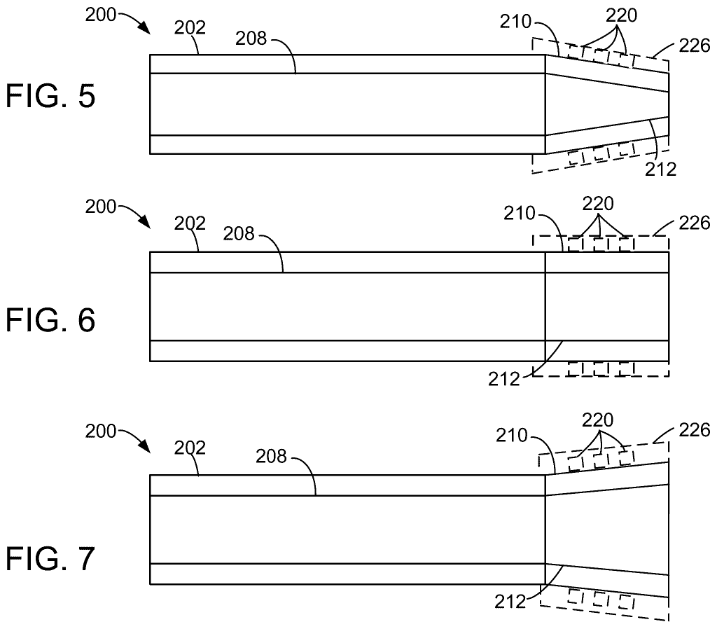

FIGS. 5-7 are schematic illustrations of a portion of the cannula system 200 showing the cannula 202 and the cannula tip 210. The cannula tip 210 (also referred to as “tip”) defines a tip lumen 212 connected to or in communication with the cannula lumen 208. In some embodiments, the tip 210 can be included in and/or integrated into the distal end 204 of the cannula 202. Alternatively, the tip 210 can be formed independent of the cannula 202 and joined (e.g., fixedly or permanently joined) to the distal end 204 of the cannula 202 during manufacturing (e.g., via an ultrasonic weld, adhesive or other chemical bond, and/or the like). In other embodiments, the tip 210 can be removably couplable to the distal end 204 of the cannula 202 (e.g., by an end user). Such an arrangement, for example, can allow different tips with different sizes, shapes, configurations, characteristics, etc. to be used with the cannula system 200 and selected based at least in part on the use and/or implementation of the cannula system 200.

In this embodiment, the tip 210 is composed of a shape memory alloy such as a nickel-titanium (nitinol) alloy. The shape memory alloy may facilitate the tip 210 switching and/or transitioning between the first configuration and the second configuration. In other examples, the tip is composed of other biocompatible materials such as stainless steel, cobalt chrome alloy, titanium, nickel-titanium alloy, tantalum, tungsten plated nickel, tungsten plated brass, polycarbonate. PEEK. PES, thermoplastic elastomers, fiber-reinforced plastic, or any other suitable biocompatible material, and/or combination(s) thereof.

The tip 210 is variable and/or otherwise configured to transition between two or more configurations. For example, in this embodiment, the cannula tip 210 is configured to transition between at least a first configuration (FIG. 5), a second configuration (FIG. 6), and a third configuration (FIG. 7). In the first configuration. (e.g., a contracted, collapsed, or compressed configuration), the tip 210 can have a first shape, size, and/or arrangement that allows the tip 210 to be inserted into and fit in a lumen of the blood vessel. In the second configuration (e.g., a first expanded configuration), the tip 210 can have a second shape, size, and/or arrangement that can allow the tip 210 to engage an inner surface of the blood vessel that defines the lumen. As such, the tip 210 in the second configuration can secure the tip 210 in a desired position within the lumen of the blood vessel, as described in further detail herein. In the third configuration (e.g., a second expanded configuration), the tip 210 can have a third shape, size, and/or arrangement that can allow the tip 210 to further expand beyond the second configuration, which in turn, can allow the tip 210 to engage the inner surface of the blood vessel as the size of the blood vessel increases due to growth and/or aging of the patient (neonate).

FIG. 5 illustrates the tip 210 and/or the optional collet 226 in a first configuration (e.g., the contracted, collapsed, or compressed configuration). For example, in the first configuration, the tip 210 and/or the optional collet 226 taper from a larger diameter at a proximal end of the tip 210 to a smaller diameter at a distal end of the tip 210. In some embodiments, the smaller diameter at the distal end of the tip 210 can match or substantially match an outside diameter of the needle 214. The distal end of the tip 210) is open allowing the needle 214 to be advanced therethrough (see e.g., FIG. 4). The tapered shape of the tip 210) in the first configuration can, for example, provide a smooth transition between the outside diameter of the needle 214 and the outside diameter of the cannula 202, which can facilitate insertion of the cannula system 200 into the relatively small and/or delicate vasculature of a neonate patient. For example, at least the distal end of the tip 210 may have a width sized to fit within a lumen of a blood vessel of the umbilical cord of a neonate when in the first configuration.

FIG. 6 illustrates the tip 210) and/or the collet 226 in the second configuration (e.g., the expanded or first expanded configuration). For example, in the second configuration, the tip 210 and/or the optional collet 226 (or at least a distal portion thereof) may have a different size (width, diameter, etc.), shape, and/or arrangement than the size (width, diameter, etc.), shape, and/or arrangement of the tip 210) and/or the optional collet 226 (or at least the distal portion thereof) in the contracted configuration (e.g., the first configuration) shown in FIG. 5. Further, the size and/or shape of the tip 210 in the second configuration may allow the tip 210) to engage and/or contact an inner surface of the vessel wall (e.g., while the optional collet 226 may be allowed to engage and/or contact an outer surface of the vessel wall). For example, in the first configuration, the tip 210) can have a first shape that is a tapered frustum and in the second configuration, the tip 210 can have a second shape that is a cylinder or that is cylindrical or substantially cylindrical having any suitable diameter. The diameter defines a width of the second shape and is larger than the width of the first shape. For example, at least the distal end of the tip 210 when in the first configuration can have a width of about 1 millimeter (mm) or less, and at least the distal end of the tip 210 when in the second configuration can have a width in a range of 2 mm to 6 mm.

FIG. 7 illustrates the tip 210) and/or the optional collet 226 in the third configuration (e.g., the further expanded or second expanded configuration). For example, in the third configuration, the tip 210) and/or the optional collet 226 (or at least a distal portion thereof) may have a different size (width, diameter, etc.), shape, and/or arrangement than the size (width, diameter, etc.), shape, and/or arrangement of the tip 210) and/or the optional collet 226 (or at least the distal portion thereof) in the first expanded configuration (e.g., the second configuration) shown in FIG. 6. In some embodiments, when in the third configuration, the distal opening of the tip 210 and/or the optional collet 226 is larger than a perimeter and/or circumference of an outer surface of the cannula 202. As such, the tip 210 and/or the optional collet 226 can taper in a direction from the distal end of the tip 210 and/or the optional collet 226 toward the proximal end of the tip 210) (e.g., connected to, coupled to, and/or extending from) the cannula 202. Similarly stated, the tip 210) and/or the optional collet 226 can be tapered in a direction that is opposite a direction of the taper of the tip 210 and/or the optional collet 226 in the first configuration. In some embodiments, the tip 210) and/or the optional collet 226 are transitioned from the first expanded configuration to the second expanded configuration as the blood vessel grows in size (e.g., as the patient ages) to ensure proper and/or desired engagement between the tip 210) (and any features positioned thereon) and the vessel wall. As such, the tip 210) and/or the collet 226 are variable between at least the configurations shown in FIGS. 5-7. Moreover, in some embodiments, the tip lumen 212 and/or the cannula lumen 208 can increase in size (e.g., diameter) when the cannula system 200 is transitioned, for example, from the first expanded configuration to the second expanded configuration. Accordingly, a flow rate through the cannula system 200 can increase as the lumens 208, 212 enlarge due to the transitioning from the first expanded configuration to the second expanded configuration. In some embodiments, increased flow rate to the patient may be desirable as the patient ages and/or grows in size.

While the cannula tip 210) and the optional collet 226 are shown in FIGS. 5-7 in three discrete configurations, it should be understood that the cannula tip 210) and the optional collet 226 can be transitioned between and/or through any number of configuration. The three discrete configurations are shown for illustrative purposes only and not limitation. For example, in some implementations, after transitioning the tip 210) to the first expanded configuration to engage the inner surface of the blood vessel, the tip 210 can be periodically adjusted (e.g., expanded) to maintain a desired degree of engagement of the blood vessel while the patient ages and/or grows in size (and thus, while the inner diameter of the blood vessel increases).

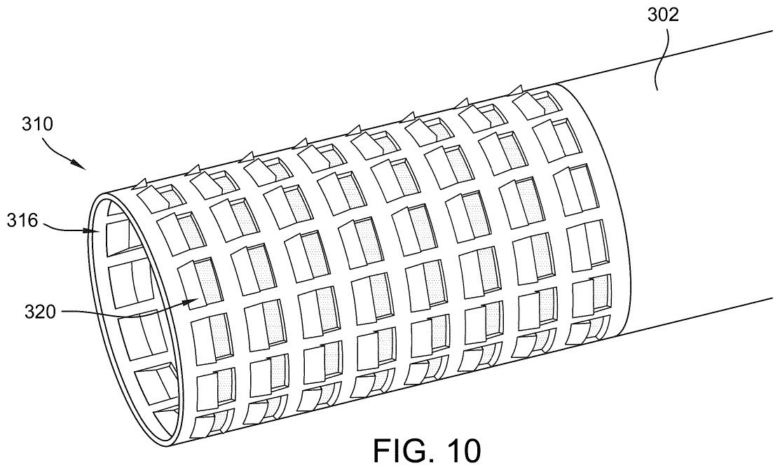

As shown in FIGS. 4-7, the tip 210 comprises and/or forms features 220 that are disposed on or formed by an outer surface of the tip 210) that are arranged to engage an inner surface of the blood vessel (e.g., when the tip 210) is in the second configuration and/or the third configuration). The features 220 may comprise ribs, barbs, bumps, scallops, protrusions, and/or the like and may facilitate the tip 210 engaging and securing to the blood vessel. For example, the features 220 may be arranged in any suitable pattern. For example, as shown in FIG. 10, the features 220 are arranged in a regular pattern (e.g. symmetric with respect to a transverse axis of the cannula 202). In other embodiments, the features 220 can be in an irregular pattern (e.g. non-symmetric with respect to a transverse axis of the cannula). In the embodiments described herein, the features 220 can be disposed on or formed by the outer surface of the tip 210 such that the features 220 facilitate engaging an inner surface of the blood vessel when the tip 210 is in the second configuration but do not hinder introduction of the tip 210 into the blood vessel when the tip 210) is in the first configuration. In other embodiments, the features 220) are omitted. In some embodiments, the features 220 can be configured to transition with or in response to the tip 210) and/or the optional collet 226 transitioning from, for example, the first (collapsed) configuration toward the second (expanded) configuration. For example, in some embodiments, the features 220 can be in a collapsed, retracted, or disengaged configuration when the tip 210) is in the first configuration and can be configured to transition to an expanded or engagement configuration when the tip is transitioned away from the first configuration. In some embodiments, the features 220 can be configured to transition in a single direction from a collapsed configuration to an expanded configuration (and not in the opposite direction—from the expanded configuration to the collapsed configuration). In some embodiments, such an arrangement can resist, limit, and/or substantially prevent the cannula system 200 being unintentionally or undesirably withdrawn or backed out of the blood vessel.

Referring back to FIG. 4, examples of using the cannula system 200 are provided below. In some embodiments, for example, the cannula system 200 may be primed with blood prior to inserting the cannula system 200 within a blood vessel of a patient. The tip 210 can be in and/or can be maneuvered to the first configuration (e.g., as shown in at least FIGS. 4 and 5) such that a distal opening of the tip 210 is sealed or plugged (e.g., by another component of the cannula system 200 extending therethrough). For example, the needle 214 may be disposed or introduced within the cannula lumen 208 such that a distal end of the needle 214 extends through and is distal to the tip 210. When the tip 210 is in the first configuration, inner wall(s) of the tip 210) (e.g., defining the tip lumen 212) can contact or otherwise engage with the needle 214 to seal the distal opening of the tip lumen 212. In addition, the needle 214 can be inserted into the cannula system 200 via the access port 218, which can similarly form a seal with an outer surface of the needle 214. In this manner, the cannula lumen 208 and the tip lumen 212 can be filled with blood and/or otherwise primed with fluid while limiting or preventing blood and/or fluid flow through the distal opening of the tip 210.

In some embodiments, the needle 214 may be disposed within the optional needle sheath 222 extending along a longitudinal axis of the cannula system 200 through the cannula lumen 208 and the tip lumen 212. For example, the needle 214 can be maneuvered into the cannula system 200 by advancing the needle 214 along a length of the needle sheath 222 from a proximal end of the needle sheath 222 to a distal end of the needle sheath 222. In some embodiments, the access port 218 (e.g., a septum seal) can be configured to facilitate advancement of the needle 214 within the needle sheath 222 from a proximal end of the cannula system 200. The inner wall(s) of the tip 210 can engage with the needle sheath 222 to seal the distal opening when the tip 210) is in or maneuvered to the first configuration. In some embodiments, the needle sheath 222 extends through the distal opening of the tip 210) such that a distal end of the needle sheath 222 is fluidly coupled to a blood source (e.g., a blood circuit, an oxygenation circuit such as the oxygenation circuit 400 described above with reference to FIGS. 1-3, and/or the like). As such, the needle sheath 222 can be configured to facilitate a flow of blood from the blood circuit distal to the tip 210 towards a proximal end of the cannula system 200 (e.g., through the distal opening of the tip 210). In some embodiments, the needle sheath 222 includes at least one outlet (e.g., channel, perforation, cavity, etc.) that fluidly couples an inner lumen of the needle sheath 222 to the cannula lumen 208 and/or the tip lumen 212. As such, the needle sheath 222 can be configured to deliver blood from the blood source to the cannula lumen 208 and/or the tip lumen 212 via the at least one outlet. In this manner, the cannula lumen 208, the tip lumen 212, and the inner lumen of the needle sheath 222 can be filled with blood and/or otherwise primed with fluid while limiting or preventing blood and/or fluid flow through the distal opening of the tip 210. In some embodiments, the cannula lumen 208, the tip lumen 212, and/or the inner lumen of the needle sheath 222 are completely filled with blood and/or fluid (e.g., a blood substitute or the like) such that air is limited or prevented from being introduced into the blood vessel of the patient. Similarly stated, the cannula lumen 208, the tip lumen 212, and/or the inner lumen of the needle sheath 222 are completely filled with blood and/or fluid and hemostatically or hermetically sealed. In some implementations, priming the cannula system 200 as described above can limit and/or substantially prevent air being introduced into the circulatory system of the patient (e.g., the neonatal patient).

In some embodiments, the needle sheath 222 is removeable. For example, the needle sheath 222 may be removed from the cannula system 200 by proximally retracting the needle sheath 222 from the distal opening of the tip 210) and, ultimately, out of the cannula 202 (e.g., via the access port 218). Alternatively, the needle sheath 222 may be removed from the cannula system 200 by distally advancing the needle sheath 222 through the distal opening of the tip 210) and, ultimately, out of the tip 210) (e.g., prior to being inserted into the patient). The needle 214 can be removed with the needle sheath 222 or can remain within the tip lumen 212 and the cannula lumen 208 while the needle sheath 222 is removed. For example, after advancing the needle 214 through the cannula system 200, the needle 214 can be maintained in a position extending through the distal opening of the tip 210 while the needle sheath 222 is removed. When the needle sheath 222 is removed, the tip 210) may transition to another shape that is more tapered or otherwise shrinks, collapses, and/or contracts the distal end of the tip 210) to a smaller diameter. In this manner, the tip 210 can engage with and/or contact an outer surface of the needle 214 (e.g., upon removal and/or retraction of the needle sheath 222) to seal the distal opening of the tip 210 against the outer surface of the needle 214.

In some embodiments, the optional guidewire 224 is positioned within the needle 214 (e.g., a lumen of the needle 214) when the needle 214 is positioned within the cannula system 200. Further, the guidewire 224 can be configured to facilitate advancement of the cannula system 200 into a blood vessel of a patient. For example, the guidewire 224 may be inserted into and disposed within a blood vessel of patient (e.g., with little to no blood flow obstruction) prior to insertion of the cannula system 200 therein. In turn, the cannula system 200 can be configured to pass over the guidewire 224 such that the distal end of the needle 214 is advanced towards the blood vessel. The distal end of the needle 214 can puncture the vessel wall and act as a dilator to increase an opening in the vessel wall created by the guidewire 224 as the needle 214 advances into the blood vessel. For example, the needle 214 can increase the opening in the vessel wall to enable insertion of the tip 210 through the opening and into the blood vessel. In some embodiments, the cannula system 200 can be advanced over the guidewire 224 and into the blood vessel until at least the tip 210 is disposed within (e.g., fully within or inside) the vessel. In some embodiments, the needle 214 dilates the opening in the vessel wall until the tip 210 can expand to the second configuration (e.g., as shown in FIG. 6). In turn, the guidewire 224 can be removed by proximally retracting the guidewire 224 into or through the needle 214.

In some embodiments, the optional collet 226 can be used to secure and/or can be used to enhance or facilitate securement of the cannula system 200 to the vessel wall. For example, the collet 226 can be used to provide additional support to the tip 210) as the features 220 included on or formed by the tip 210) attach and/or couple to the vessel wall. As previously mentioned, the features 220 can be configured to engage the inner surface of the blood vessel to secure a portion of the cannula system 200 in the blood vessel. In some embodiments, the collet 226 can be configured to engage with an outer surface of the blood vessel such that an annular wall of the blood vessel is sandwiched between the of the tip 210) (having the features 220) and an inner surface of the collet 226. Put differently, the features 220 can be secured to an inner surface of the blood vessel while the collet 226 can be secured to and/or otherwise engage or contact an outer surface of the blood vessel. As such, the collet 226 can be tightened or contracted to reduce an inner radial dimension of the collet 226, thereby placing an inner surface of the collet 226 in contact with the outer surface of the vessel wall. As such, the structural support associated with the collet 226 can facilitate an increased or more secure engagement between the features 220) and the inner surface of the blood vessel. In some embodiments, the collet 226 surrounds the umbilical cord of a neonate when the tip 210 is disposed within a blood vessel in the umbilical cord. In such embodiments, the collet 226 can be tightened or contracted to exert a force in a radial direction on the umbilical cord to sandwich the umbilical cord and the vessel wall between the tip 210 and the collet 226. In some embodiments, the collet 226 circumferentially surrounds the tip 210) and/or an outer surface of the cannula 202. For example, the collet 226 can at least partially circumferentially surround a rigid section of the cannula 202.

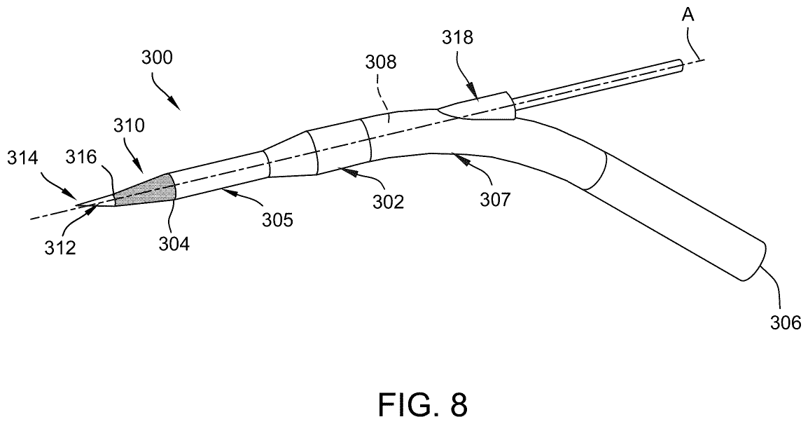

FIGS. 8-10 depict at least a portion of a cannula system 300 according to embodiments of the present disclosure. The cannula system 300 may be integrated into the extracorporeal support system 10) (and/or the oxygenation circuit 400) thereof) described above with reference to FIGS. 1-3. In some cases, the cannula may be integrated into the cannula insertion system described with reference to the '270 publication.

The cannula system, generally indicated at 300, may be arranged for cannulating a blood vessel. The cannula system 300 can be structurally and/or functionally similar to other cannula systems described herein including, for example, the cannula system 200 described above with reference to FIGS. 1-7. The cannula system 300 includes a cannula 302 (e.g., structurally and/or functionally similar to other cannulas described herein including, for example, the cannula 202 described above with reference to FIGS. 1-7) having a distal end 304 and a proximal end 306. The cannula 302 defines a cannula lumen 308 extending through the cannula 302 from the proximal end 306 to the distal end 304. In the example shown in FIGS. 8-10, the cannula 302 includes a rigid section 305 extending proximally from the distal end 304, and a flexible section 307 extending from the rigid section 305 to the proximal end 306. Suitably, the cannula 302 is composed of biocompatible materials such as stainless steel, cobalt chrome alloy, titanium, nickel-titanium alloy, tantalum, tungsten plated nickel, tungsten plated brass, polycarbonate. PEEK. PES, thermoplastic elastomers, fiber-reinforced plastic, or any other suitable biocompatible material, and/or combination(s) thereof.

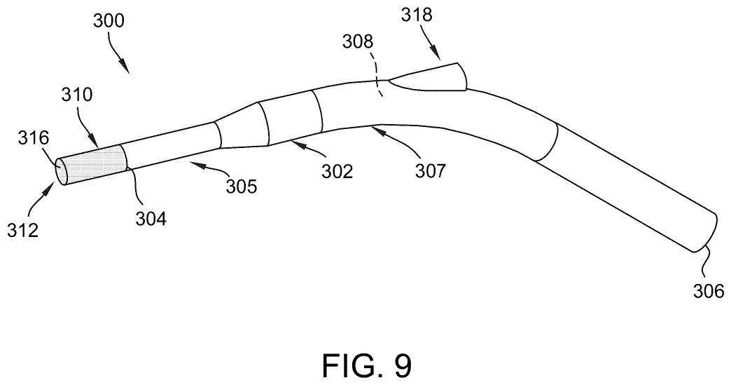

The cannula system 300 includes a tip 310 (e.g., structurally and/or functionally similar to other tips described herein including, for example, the tip 210 described above with reference to FIGS. 1-7) on the distal end 304 of the cannula 302. The tip 310 (also referred to as “cannula tip”) defines a tip lumen 312 connected to the cannula lumen 308. The tip 310 is variable between a first configuration (shown in FIG. 8) and a second configuration (shown in FIGS. 9 and 10). In the first configuration, the tip 310) can have a first shape, size, and/or arrangement that allows the tip 310 to be inserted into and fit within a lumen of the blood vessel. In the second configuration, the tip 310 can have a second shape, size, and/or arrangement that allows the tip 310) to engage an inner surface of the blood vessel that defines the lumen. As such, the tip 310) in the second configuration can secure the tip 310 in a desired position within the lumen of the blood vessel, as described in further detail herein.

The cannula system 300 includes a needle 314 (e.g., structurally and/or functionally similar to other needles described herein including, for example, the needle 214 described above with reference to FIG. 4) sized and configured to extend through the cannula lumen 308 and the tip lumen 312. The tip 310) defines a distal opening 316 arranged for the needle 314 to extend through and engage or puncture the blood vessel. The needle 314 can be a relatively rigid and straight (or substantially straight) elongate tube or member having a sharpened and/or beveled distal tip allowing the needle 314 to puncture tissue, blood vessels, and/or the like.

In the example shown in FIGS. 8-10, the cannula 302 includes a septum seal 318 that is proximal of the tip 310) and is arranged for the needle 314 to enter into or exit the cannula lumen 308. The septum seal 318 is located on the flexible section 307 of the cannula 302. More specifically, the septum seal 318 can be in a position along the cannula 302 that is substantially aligned with the distal opening 316 of the tip 310. For example, as shown in FIG. 8, the septum seal 318, at least a portion of the cannula lumen 308, and the tip lumen 312 are aligned such that an axis A extends therethrough. Thus, the septum seal 318 facilitates the insertion of the relative rigid, straight needle 314 through the cannula system 300) and allows simple removal of the needle 314, for example, without a slit port.

The variable cannula tip 310 is described in further detail below with reference to FIGS. 8-10. In some embodiments, the tip 310) can be included in and/or integrated into the distal end 304 of the cannula 302. Alternatively, the tip 310 can be formed independent of the cannula 302 and joined (e.g., fixedly or permanently joined) to the distal end 304 of the cannula 302 during manufacturing (e.g., via an ultrasonic weld, adhesive or other chemical bond, and/or the like). In other embodiments, the tip 310) can be removably couplable to the distal end 304 of the cannula 302 (e.g., by an end user). Such an arrangement, for example, can allow different tips with different sizes, shapes, configurations, characteristics, etc. to be used with the cannula system 200 and selected based at least in part on the use and/or implementation of the cannula system 200.

As described above, the tip 310 is variable between a first configuration and a second configuration. In the first configuration, the tip 310 can have a first shape, size, and/or arrangement that allows the tip 310 to be inserted into and fit in a lumen of the blood vessel. In the second configuration, the tip 310 can have a second shape, size, and/or arrangement that allows the tip 310 to engage an inner surface of the blood vessel that defines the lumen. In some embodiments, the second shape (e.g., when the tip 310 is in the second configuration) may be a different size and/or geometric shape than the first shape (e.g., when the tip 310 is in the first configuration). For example, the tip 310 is a perforated, flexible sleeve or stent. In the first configuration, the tip 310 can have a first shape that is a tapered frustum and in the second configuration, the tip 310 can have a second shape that is cylindrical or substantially cylindrical having any suitable diameter. The diameter defines a width of the second shape and is larger than the width of the first shape. For example, the first shape may have a width about 1 mm or less, and the second width of the second shape may be in a range of 3 mm to 6 mm. The tip 310 may have length in a range of 6 mm to 11 mm. In other embodiments, the tip 310 may have other shapes. For example, in some embodiments, the tip 310 (e.g., in the second configuration) may be cuboid, spherical, rectangular cuboid, and/or any suitable shape.

In this embodiment, the tip 310 is composed of a shape memory alloy such as a nickel-titanium (nitinol) alloy. The shape memory alloy may facilitate the tip 310 switching and/or transitioning between the first configuration and the second configuration. In other examples, the tip is composed of other biocompatible materials such as stainless steel, cobalt chrome alloy, titanium, nickel-titanium alloy, tantalum, tungsten plated nickel, tungsten plated brass, polycarbonate. PEEK. PES, fiber-reinforced plastic, or any suitable combination(s) thereof.

As seen in FIG. 8, in the first configuration, the tip 310 tapers from a larger diameter at the proximal end 306 to a smaller diameter that matches the outside diameter of the needle at the distal end 304. The smaller diameter defines a minimum width associated with the tip 310 in the first configuration. The tip 310 defines the distal opening 316 arranged for the needle 314 to extend through and engage or puncture the blood vessel at the distal end. The tapered shape of the tip 310 in the first configuration provides a smooth transition between the outside diameter of the needle 314 and the outside diameter of the cannula 302.

In the example shown in FIGS. 8-10, the tip 310 comprises and/or forms features 320 (e.g., structurally and/or functionally similar to other features described herein including, for example, features 320 described above with reference to FIG. 4-7) that are disposed on an outer surface of the tip 310 and are arranged to engage an inner surface of the blood vessel (e.g., when the tip 310 is in the second configuration). The features 320 may comprise ribs, barbs, bumps, scallops, protrusions, and/or the like and may facilitate the tip 310 engaging and securing to the blood vessel. For example, the features 320 may be arranged in any suitable pattern. For example, as shown in FIG. 10, the features 320 are arranged in a regular pattern (e.g. symmetric with respect to a transverse axis of the cannula 302). In other embodiments, the features 320 can be in an irregular pattern (e.g. non-symmetric with respect to a transverse axis of the cannula). In the embodiments described herein, the features 320 can be disposed on or formed by the outer surface of the tip 310 such that the features 320 facilitate engaging an inner surface of the blood vessel when the tip 310 is in the second configuration but do not hinder introduction of the tip 310 into the blood vessel when the tip 310 is in the first configuration. In other embodiments, the features 320 are omitted.

Although not shown in FIGS. 8-10, in some embodiments, the cannula system 200 can include a retainer configured to secure and/or at least temporarily maintain the tip 310 in the first configuration or the second configuration. For example, the retainer may be a separate component that engages the tip 310 when the tip 310 is in the first configuration or the second configuration. The retainer may be removed or adjusted to switch or transition the tip 310 (or otherwise allow the tip 310 to switch or transition) between the first configuration and the second configuration. In some embodiments, the retainer can be a bias member, a latch, a catch, a sleeve, a sheath, and/or any other suitable retainer that can be removably coupled to the tip 310 and/or the cannula 302. Alternatively, the retainer can be incorporated into and/or can be a feature or characteristic of the tip 310 and/or the cannula 302.

In some embodiments, the tip 310 may be in a neutral state, position, etc. when in the first configuration or the second configuration and configured to transition in response to an exerted force that may retain or hold the tip 310 in the second configuration of the first configuration, respectively (or vice versa). Suitably, the retainer may retain the tip 310 in the first configuration and/or the second configuration. In some embodiments, the retainer is omitted.

In some embodiments, the tip 310 is in the second configuration when in the neutral state, and the retainer is incorporated with the needle 314. As such, features on the needle 314 can engage and retain the tip 310 in the first configuration, which is a collapsed or restrained state of the tip 310 (as shown in FIG. 8). When the needle 314 is removed, the restraint on the tip 310 is removed, and the tip 310 is allowed to transition to the second configuration (e.g., expands to the second shape and/or size, as shown in FIGS. 9 and 10).

In some embodiments, the tip 310 is in the second configuration when in the neutral state, and the retainer is a sheath, sleeve, and/or the like (not shown). For example, such a sheath can be positioned over the tip 310 to secure the tip 310 in the first configuration (e.g., a collapsed or restrained state or shape. The sheath can be removed from the tip 310 when the cannula 302 and the tip 310 are in a desired position within the blood vessel. When the sheath is removed, the restraint on tip 310 is removed, and the tip 310 is allowed to transition to the second configuration (e.g., expands to the second shape and/or size).

In some embodiments, the tip 310 is in the second configuration when in the neutral state, and the retainer is a restraining filament (not shown) that engages one or more portions of the tip 310. When the filament is removed, the restraint on the tip 310 is removed, and the tip 310 is allowed to transition to the second configuration (e.g., expands to the second shape and/or size). In such embodiments, the filament can be disposed in the tip lumen 312 and releasably attached to the inner surface of the tip 310, or can be releasably attached to an outer surface of the tip 310.

In some embodiments, the retainer is not needed. For example, in such embodiments, the tip 310 is in the first configuration when in a neutral state. When the tip 310 is in position within the blood vessel, a bladder or dilator (not shown) is inflated inside of the tip 310 and the bladder and/or dilator transitions or expands the tip 310 (e.g., by plastic deformation) to the second shape. The bladder or dilator can then be removed from the cannula system 200.

In some embodiments, the retainer is incorporated into the tip 310 and the cannula 302 such that the tip 310 is held in the first shape by the position of the tip 310 relative to the cannula 302. For example, twisting of the tip 310 or at least a distal end of the tip 310 relative to the cannula 302 can cause at least the distal end of the tip 310 to be in the first configuration. After positioning the tip 310 within the blood vessel, the tip 310 (or at least the distal end of the tip 310) can be twisted relative to the cannula 302 (e.g., in an opposite direction) to change or transition the tip 310 to the second configuration (e.g., to expand at least the distal end portion of the tip 310).

In some embodiments, at least the tip 310 can be formed from a superelastic and/or shape memory alloy (e.g., nickel-titanium alloy such as Nitinol®). The tip 310 can be biased (e.g., heat set) in the first configuration or the second configuration and the superelastic material can allow the tip to transition. As described above, the cannula system 300 can include any suitable retainer configured to at least temporarily maintain the tip 310 in an unbiased state (e.g., the first configuration), and the tip 310 can be allowed to transition to the biased state (e.g., the second configuration) when the retainer is removed. In other embodiments, positioning of the tip 310 in the blood vessel (e.g., in the flow of blood, exposed to normal body temperatures, etc.) can cause phase change associated with the shape memory alloy, which in turn, can cause the tip 310 to transition from the first configuration to the second configuration.

In some embodiments, the tip 310 and at least a portion of the cannula 302 (e.g., the flexible section) are a single piece. The tip 310 and the cannula 302 may be formed from the same material (e.g., Nitinol®) to facilitate operation of the cannula 302 and the tip 310. In further embodiments, a flexible section of the cannula 302 proximal to the tip 310 is a deformable stent structure and facilitates the tip 310 and/or an introduced element such as a balloon, dilator, bladder, etc. to expand in size. The cannula system 300 may include a deformable liquid tight cover around or inside of the deformable cannula 302 (e.g., the deformable stent structure) to affect a liquid tight structure.

In one embodiment, an exterior collet mechanism (not shown in FIGS. 8-10) is incorporated into the cannula 302 and clamps over the vessel tissue around the cannula to facilitate securing the cannula in position, in conjunction with the tip 310). In some embodiments, the exterior collet mechanism can be structurally and/or functionally similar to other collets described herein including, for example, the collet 226 described above with reference to FIG. 4-7.

As described above, the cannula systems described herein can be used to penetrate a targeted blood vessel, expand an opening in the vessel wall, insert a portion of a cannula into the blood vessel through the created opening, and secure the cannula to the vessel wall via a cannula tip (or otherwise in response to the cannula tip transitioning from a first configuration to a second configuration).

In some embodiments, the cannula system can include a needle assembly. The needle assembly can include a needle and, optionally, a needle sheath. For example, the needle is configured to extend through an opening in a cannula tip on a distal end of a cannula, and pierce a blood vessel thereby creating a passage into the vessel. The tip and the cannula can be inserted through the passage into the lumen defined by the blood vessel. In some embodiments, the needle passes over a guidewire to facilitate advancement of the tip and the cannula into the lumen defined by the blood vessel.

Suitably, the cannula systems described herein can be used with or without a dilator assembly. For example, the needle can be housed in the cannula tip instead of within a dilator assembly. Also, the tip can be arranged to facilitate manipulation of the needle and facilitate insertion of the cannula into the blood vessel lumen. In addition, the tip can be configured to transition (e.g., expand or otherwise reconfigure) to secure the cannula in position and create a larger opening in the blood vessel after the needle pierces the wall of the blood vessel. For example, the tip can be transitioned from a first configuration (e.g., a contracted configuration having a first shape, size, width, etc.) that allows the tip to fit in the lumen of the blood vessel to a second configuration (e.g., an expanded configuration having a second shape, size, width, etc.) that allows the tip to engage an inner surface of the blood vessel and secure the tip in a desired position within the lumen of the blood vessel.

The cannula systems described herein are configured to fluidly connect to the cannulated blood vessel at one end and to a circulation system and/or oxygenation system at another end. The cannula and the tip can define a lumen that extends the length of the cannula and the tip.

While some of the cannula tips are described herein as being transitioned between a first configuration and a second configuration, it should be understood that the embodiments described herein can include a cannula tip configured to transition between two or more configurations. For example, a cannula tip can be configured to transition from a collapsed or delivery configuration to any number of expanded configurations. In such embodiments, as the patient (neonate) grows and/or ages, the cannula tip can be transitioned, for example, from a first expanded configuration to a second expanded configuration to ensure proper and/or desired engagement with the vessel wall and/or to increase a flow rate from the circulation system to the vessel. In some embodiments, any of the cannula tips described herein can include any number of expanded configurations allowing the cannula tip to exert a desired force against an inner surface of the blood vessel to connect and/or secure the cannula system to the blood vessel.

Moreover, while embodiments have been shown as including the variable tip at a distal end of the cannula, in some embodiments, at least a portion of the variable structure can extend along a portion of the cannula, thereby allowing at least that portion of the cannula to transition between any number of configurations. For example, embodiments described herein can be used in extracorporeal support systems configured to support neonate patients. In such implementations, the cannula systems described herein can be used to cannulate blood vessels of the umbilical cord of the neonate, which are very small and limit the size of the cannula system as well as the flow rate through the cannula system (particularly in cases of extreme prematurity). As such, portions of the cannula that are inserted into the blood vessel may have a reduced outer diameter (and therefore, a reduced inner diameter). As described above, as the neonate grows and/or ages, the inner diameter of the blood vessel increases, and the cannula tips described herein can be expanded to maintain a desired engagement with the vessel walls. In addition, the tip lumen can expand allowing an increased flow rate therethrough.

In some embodiments, the expansion of the tip can cause expansion of at least a portion of the cannula such as, for example, at least a portion of the cannula lumen extending through a portion of the cannula disposed in the vessel. For example, as shown in FIGS. 8 and 9, the substantially rigid section 305 of the cannula 302 can have a reduced diameter (e.g., relative to the substantially flexible section 307), making the substantially rigid section 305 suitable for disposal in the blood vessel. In some instances, as the tip 310 is expanded, at least a portion of the cannula lumen 308 that extends through the substantially rigid section 305 of the cannula 302 may also be expanded, stretched, increased, dilated, etc. In some embodiments, the substantially rigid section 305 of the cannula 302 or any other suitable portion of the cannula 302 can include a variable or expandable structure that can allow such portions of the cannula 302 to at least partially expand (e.g., in response to or based on the increase in size of the blood vessel as the neonate grows). The variable structure can be similar to or different from structure of the variable tip 310. Accordingly, the embodiments described herein can be configured to selectively allow an increased flow rate through the cannula system as the neonate grows.

In some embodiments, the cannula systems described herein provide a method for a clinician to securely attach the cannula to the cannulated region such as, without limitation, a blood vessel. For example, a needle can be passed though a septum seal of the cannula system, advanced through a cannula lumen and out of a cannula tip on a distal end of the cannula. The cannula tip can have a first configuration in which the tip tapers from a larger diameter at the distal end to a smaller diameter that matches or substantially matches the outside diameter of the needle. The tapered shape can provide a smooth transition between the needle outside diameter and the cannula outside diameter. During the cannulation procedure, the needle is used to penetrate the vessel wall. The cannula is advanced such that the distal end of the cannula tip passes through the opening made in the vessel wall and into the lumen of the blood vessel. When at least the cannula tip is inside of the vessel, the needle can be retracted into the lumen defined by the tip or the cannula, or can be retracted and withdrawn from the cannula system. The cannula system (or at least the tip and a portion of the cannula) can then be advanced into a desired position in the blood vessel (e.g., until at least a portion of a rigid section of the cannula is inside the lumen of the vessel). When the cannula is in a desired position, the method includes transitioning or switching the cannula tip on the distal end of the cannula from the first configuration (e.g., having a tapered shape) to a second configuration. The tip in the second configuration can be such that the tip or at least a portion of the tip has a second width that is larger than the first width associated with the distal end of the tip when the tip is in the first configuration. The tip in the second configuration is shaped and sized to engage an inner surface of the blood vessel that defines the lumen. The tip in the second configuration (e.g., an expanded, second shape, size, etc.) is such that at least a portion of the tip presses into the inner surface of the blood vessel forming a connection therebetween. In addition, the tip in the second configuration provides an open path from the vessel lumen to the cannula lumen.

In some implementations, the needle can be removed from the tip lumen and/or the cannula lumen when the tip is in the second, expanded configuration. The cannula system can include a septum seal configured to receive the needle and further configured to form a seal upon removal of the needle from the cannula.

Although preferred embodiments of the invention have been described using specific terms, such description is for illustrative purposes only, and it is to be understood that changes and variations may be made without departing from the spirit or scope of the following claims.

The specific terminology used herein is for the purpose of describing particular embodiments and/or features or components thereof and is not intended to be limiting. While various schematics, embodiments, and/or implementations have been described above, it should be understood that they have been presented by way of example only, and not limitation. Various modifications, changes, and/or variations in form and/or detail may be made without departing from the scope and/or spirit of the disclosure and/or without altering the function and/or advantages thereof unless expressly stated otherwise. Likewise, while embodiments (and/or features, components, configurations, aspects, etc. thereof) may be described above in the context of certain implementations, it should be understood that such implementations are presented by way of example only, and not limitation. Any of the embodiments (and/or features, components, configurations, aspects, etc. thereof) can be used in, and/or adapted for use in, other implementations unless expressly stated otherwise. Functionally equivalent embodiments, implementations, and/or methods, in addition to those described herein, will be apparent to those skilled in the art from the foregoing descriptions and are intended to fall within the scope and/or spirit of the disclosure.

Where schematics, embodiments, and/or implementations described above indicate certain components arranged in certain orientations, configurations, or positions, the arrangement of components may be modified. Although various embodiments have been described as having particular features, configurations, and/or combinations of components, other embodiments are possible having a combination of any features, configurations, and/or components from any of embodiments described herein, except mutually exclusive combinations. The embodiments described herein can include various combinations and/or sub-combinations of the functions, components, configurations, and/or features of the different embodiments described.

The specific configurations of the various components can also be varied. For example, the size and specific shape of the various components can be different from the embodiments shown, while still providing the functions as described herein. More specifically, the size and shape of the various components can be specifically selected for a desired or intended usage. Thus, it should be understood that the size, shape, and/or arrangement of the embodiments and/or components thereof can be adapted for a given use unless the context explicitly states otherwise.

Where methods described above indicate certain events, steps, and/or procedures occurring in certain order, the ordering of certain events, steps, and/or procedures may be modified. Additionally, certain of the events, steps, and/or procedures may be performed concurrently in a parallel process, when possible, as well as performed sequentially as described above. While methods have been described as having particular steps and/or combinations of steps, other methods are possible having a combination of any steps from any of methods described herein, except mutually exclusive combinations and/or unless the context clearly states otherwise.

### INCORPORATION BY REFERENCE

The entire contents of all patents, published patent applications, and other references cited herein are hereby expressly incorporated herein in their entireties by reference.

What is claimed is:

1. A cannula system for cannulating a blood vessel, the cannula system comprising:
a cannula having a distal end and a proximal end, the cannula defining a cannula lumen; and
a tip on the distal end of the cannula, the tip defining a tip lumen connected to the cannula lumen, wherein the tip is variable between a first shape and a second shape, the first shape has a first width sized to fit in a lumen of the blood vessel, the second shape has a second width larger than the first width and sized to engage an inner surface of the blood vessel defining the lumen and secure the tip in the lumen of the blood vessel.

2. The cannula system of claim 1, wherein the cannula includes a rigid section extending from the distal end and a flexible section extending from the rigid section to the proximal end.

3. The cannula system of claim 1, further comprising:
a needle sized to extend through the cannula lumen and the tip lumen, and wherein the tip defines a distal opening arranged for the needle to extend out of the tip lumen and engage the blood vessel.

4. The cannula system of claim 3, wherein the cannula includes a septum seal that is proximal of the tip and is arranged for the needle to enter into the cannula lumen.

5. The cannula system of claim 1, wherein the tip is a perforated, flexible sleeve, and wherein the first shape of the tip is a frustum, and the second shape of the tip is a cylinder.

6. The cannula system of claim 1, wherein the tip comprises features that are disposed on an outer surface of the tip and are arranged to engage the inner surface of the blood vessel.

7. The cannula system of claim 6, wherein the features are arranged in an irregular pattern on the outer surface of the tip.

8. The cannula system of claim 1, wherein at least one of the cannula and the tip is composed of a nickel-titanium shape memory alloy.

9. The cannula system of claim 1, further comprising:
a retainer configured to secure the tip in the first shape or the second shape, wherein the retainer is configured to be removed or adjusted to switch the tip between the first shape and the second shape.

10. The cannula system of claim 1, wherein the cannula is composed of stainless steel, cobalt chrome alloy, titanium, nickel-titanium alloy, tantalum, tungsten plated nickel, tungsten plated brass, polycarbonate, PEEK, PES, fiber-reinforced plastic, or a combination thereof.

11. The cannula system of claim 1, wherein the cannula system is configured to be connected to an oxygenation circuit of an extracorporeal support system.

12. The cannula system of claim 1, wherein the blood vessel is at least one of a vein or an artery of an umbilical cord of a neonate.

13. A method of cannulating a blood vessel in a tissue, the method comprising:
inserting a needle into the blood vessel to form a passage in the blood vessel, wherein the needle extends through a tip on a distal end of a cannula;
inserting the tip and the cannula through the passage into a lumen defined by the blood vessel; and
transitioning the tip from a first shape to a second shape, the first shape has a first width sized to fit in the lumen of the blood vessel, the second shape has a second width larger than the first width and sized to engage an inner surface of the blood vessel defining the lumen and secure the tip in the lumen of the blood vessel.

14. The method of claim 13, further comprising:
inserting the needle through a septum seal of the cannula and into a cannula lumen.

15. The method of claim 13, further comprising:
removing the needle from a cannula lumen when the tip is in the second shape.

16. An extracorporeal system for supporting a neonate, the system comprising:
a chamber configured to receive a neonate:
an oxygenation circuit including an oxygenator; and
a cannula system for cannulating a blood vessel, the cannula system including a cannula and a tip coupled to a distal end of the cannula, the cannula defining a cannula lumen, the tip defining a tip lumen connected to the cannula lumen, wherein the tip is variable between a first shape and a second shape, the first shape has a first width sized to fit in a lumen of the blood vessel, the second shape has a second width larger than the first width and sized to engage an inner surface of the blood vessel defining the lumen and secure the tip in the lumen of the blood vessel.

17. The system of claim 16, further comprising:
a needle sized to extend through the cannula lumen and the tip lumen.

18. The system of claim 17,
wherein the cannula includes a septum seal that is proximal of the tip and is arranged for the needle to enter into the cannula lumen, and the tip defines a distal opening arranged for the needle to extend therethrough to engage the blood vessel.

19. The system of claim 16, wherein the tip is a perforated, flexible sleeve.

20. The system of claim 16, wherein the first shape of the tip is a frustum, and the second shape of the tip is a cylinder.
